(19) United States
(12) Patent Application Publication
(10) Pub. No.: US 2007/0003461 A1
(43) Pub. Date: Jan. 4, 2007

(54) Multi-functional child care storage having a steam sterilizing compartment

(75) Inventor: Young Jae Kim, Seoul (KR)

Correspondence Address: BACON & THOMAS, PLLC, 625 SLATERS LANE, FOURTH FLOOR, ALEXANDRIA, VA, 22314, US

(73) Assignee: DAEWOO ELECTRONICS Corporation, Seoul (KR)

(21) Appl. No.: 11/377,320

(22) Filed: Mar. 17, 2006

(30) Foreign Application Priority Data
Jul. 1, 2005 (KR) 10-2005-0059015

(51) Int. Cl. A61L 2/07

(52) U.S. Cl. 422/299

(57) ABSTRACT

A multi-functional child care storage includes a plurality of functional compartments. The plurality of the functional compartments includes a steam sterilizing compartment for sterilizing an infant product loaded therein. The steam sterilizing compartment has a sterilizing space arranged in the main body for sterilizing the stored infant product; an evaporation vessel arranged inside the sterilizing space for containing water therein; a heater installed under the evaporation vessel for heating the evaporation vessel to evaporate the water contained therein into steam; an exhaust outlet through which air in the sterilizing space is exhausted to an outside; a blower for generating an air flow for exhausting the air in the sterilizing space through the exhaust outlet; and a control unit for controlling the blower and the heater to start, suspend or resume operations thereof depending on a temperature of the heater.

### FIELD OF THE INVENTION

The present invention relates to a multi-functional child care storage having a steam sterilizing compartment; and, more particularly, to a multi-functional child care storage having a steam sterilizing compartment for sterilizing infant products stored therein with steam and then removing moisture from the sterilized infant products through hot air heated by a heater.

### BACKGROUND OF THE INVENTION

A refrigerator is generally used to keep foods or beverages in a refrigerated or frozen state for a long time or to cool them rapidly. The refrigerator includes a freezer compartment and refrigerator compartment opened and closed by respective front doors. To supply cooled air to the freezer and refrigerator compartments, the refrigerator further includes a compressor, a condenser, a capillary tube and a refrigerating machine to perform a cooling cycle. In the cooling cycle, the compressor compresses a coolant at a high temperature and pressure to provide the compressed coolant to the condenser. The condenser releases heat from the compressed coolant to convert it into a low-temperature and low-pressure liquid state, which is then converted into a low-temperature and high-pressure liquid state while the coolant passes through the capillary tube. The low-temperature high-pressure liquid coolant is then directed to the refrigerating machine installed at a rear side of the freezer compartment to be sent to the refrigerating machine, evaporated and converted into a low-pressure coolant while it passes through a coolant pipe of the refrigerating machine, thereby lowering temperatures of the freezer and refrigerator compartments.

Recently, with a rise in the standard of living, there have been increasing demands for diversified types of special-purpose refrigerators. To meet such needs, for example, a kimchi refrigerator equipped with an evaporation pipe and a heating wire for ripening kimchi has been developed and widely available. There has also been developed a cosmetic cooler having a small storage compartment which is capable of keeping cosmetics at a low temperature by cooling the storage compartment using a thermoelectric element.

Although various special-purpose or multi-purpose refrigerators have been developed, special-purpose storages for baby or child care have rarely been developed.

If infant foods and/or infant products such as powdered milk, breast milk and medicines are stored in conventional storage means, there may be various problems or inconveniences. For example, if infant foods and/or infant products are stored in a conventional household refrigerator, they will be soaked with odors of other foods stored together in the refrigerator. As a result, the quality of the infant foods and/or infant products will be deteriorated and even hygienic problems may be caused. When a microwave oven is used to warm up, in particular, reconstituted milk, it is difficult to maintain the warmed-up reconstituted milk at a desired temperature for a long period of time. However, it is expensive to purchase a heating cabinet for the sole purpose of warming up reconstituted milk or storing warmed-up reconstituted milk. Furthermore, since the conventional heating cabinets are designed to be used in, for example, a hospital, pharmacy or restaurant, it is not suitable for use in home for the purpose of child care.

In addition, because an ordinary household sterilizer is designed to sterilize kitchenware such as dishware or utensils, it is improper to use the household sterilizer for sterilizing infant products such as a baby bottle, gauze handkerchief and toy for hygienic reasons.

Therefore, for the purpose of child care, freed from the conception of conventional electronic appliances designed for housewives or adults, it is necessary to develop a multi-functional storage product which is hygienic, and can store infant foods and/or infant products in various ways according to their uses and functions. Further, it is also necessary to develop a multi-functional storage product that can be easily assemblable by designing its structure less complicated while maintaining the multiple functions thereof.

### SUMMARY OF THE INVENTION

It is, therefore, an object of the present invention to provide a multi-functional child care storage having a steam-sterilizing compartment, wherein the steam sterilizing compartment sterilizes infant products stored therein with steam and then removes moisture from the sterilized infant products through hot air heated by a heater.

In accordance with the present invention, there is provided multi-functional child care storage including a plurality of functional compartments, the plurality of the functional compartments including a steam sterilizing compartment for sterilizing an infant product loaded therein, wherein the steam sterilizing compartment includes: a sterilizing space defined for sterilizing the stored infant product; an evaporation vessel arranged inside the sterilizing space for containing water therein; a heater installed under the evaporation vessel for heating the evaporation vessel to evaporate the water contained therein into steam; an exhaust outlet through which air in the sterilizing space is exhausted; a blower for generating an air flow for exhausting the air in the sterilizing space through the exhaust outlet to the outside; and a control unit for controlling the blower and the heater to start, suspend or resume operations thereof depending on a temperature of the heater.

### BRIEF DESCRIPTION OF THE DRAWINGS

The above and other objects and features of the present invention will become apparent from the following description of preferred embodiments, given in conjunction with the accompanying drawings, in which:

FIG. 1 is a perspective view of a multi-functional child care storage having a steam-sterilizing compartment in accordance with the present invention, wherein all doors thereof are opened;

FIG. 2 shows a front sectional view of the multi-functional child care storage in accordance with the present invention;

FIG. 3 illustrates a front sectional view of the steam-sterilizing compartment in accordance with the present invention;

FIG. 4 presents an exploded sectional view of the steam-sterilizing compartment in accordance with the present invention;

FIG. 5 depicts a block diagram illustrating an operation unit, a temperature sensor, a control unit, a heater and a blower in the steam-sterilizing compartment in accordance with the present invention; and

FIG. 6 represents a flow chart showing an operation of the steam-sterilizing compartment in accordance with the present invention.

### DETAILED DESCRIPTION OF THE PREFERRED EMBODIMENT

Hereinafter, a preferred embodiment of the present invention will be described in detail with reference to the accompanying drawings so that they can be readily carried out by those skilled in the art.

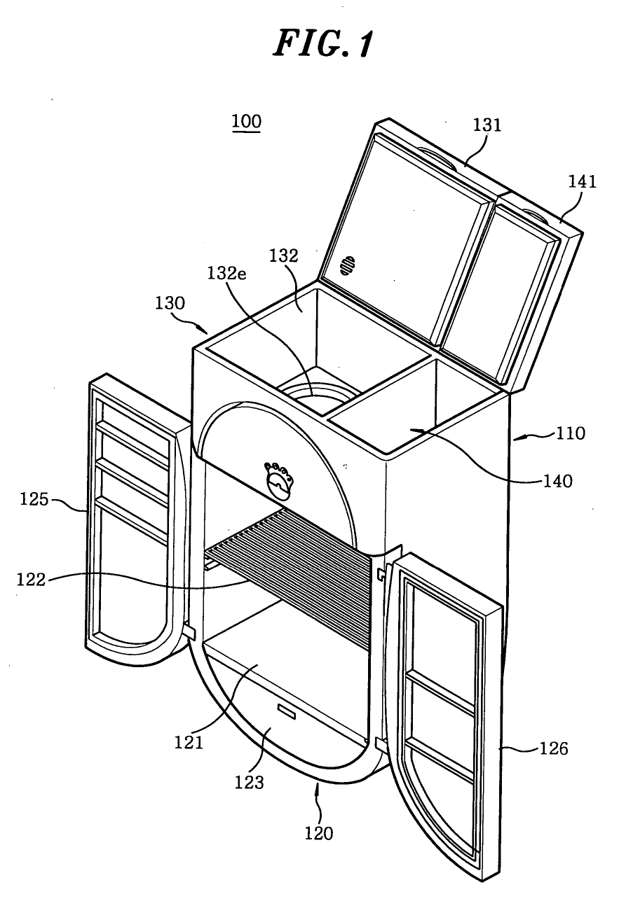

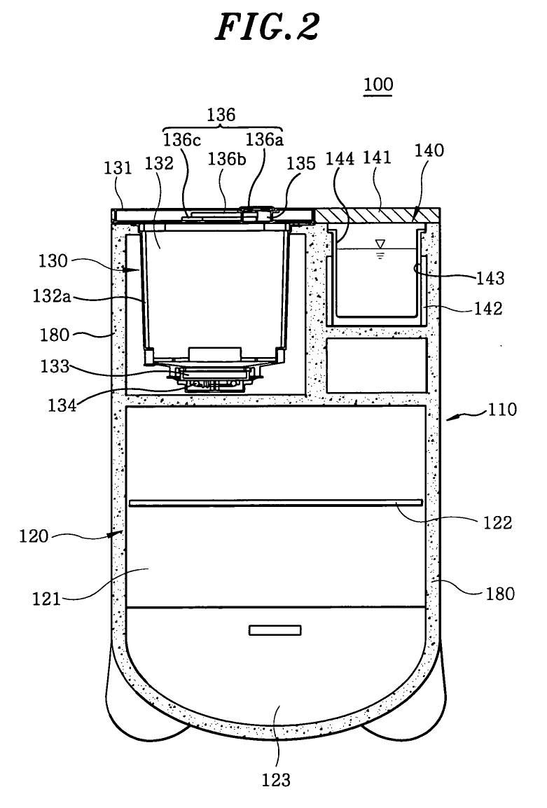

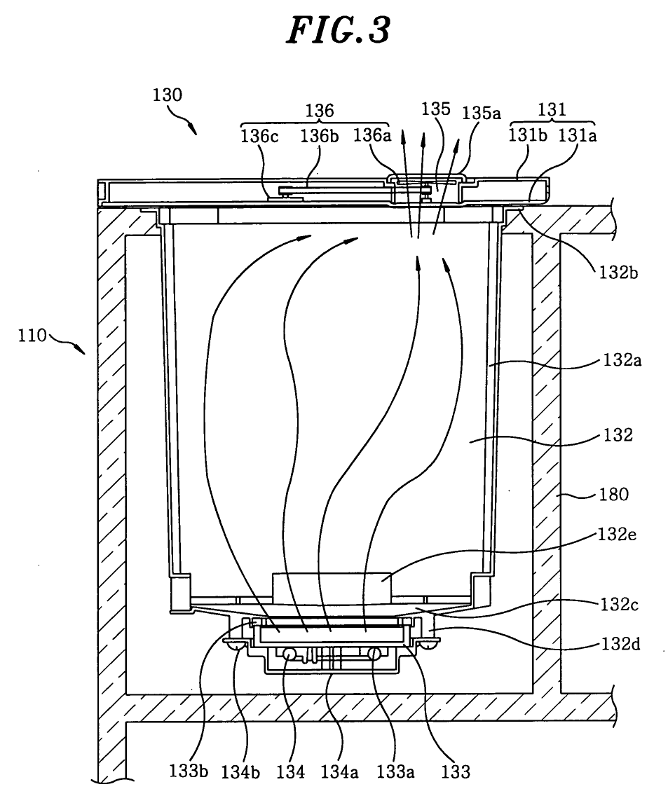

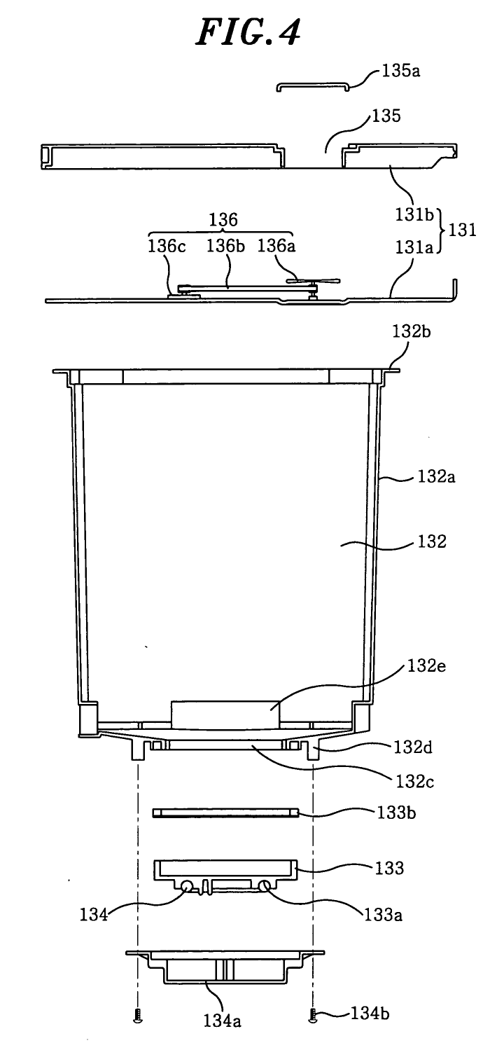

FIG. 1 is a perspective view of a multi-functional child care storage having a steam-sterilizing compartment in accordance with the present invention, wherein all doors thereof are opened. Further, FIG. 2 is a front sectional view of the multi-functional child care storage in accordance with the present invention. In addition, FIGS. 3 and 4 are a front sectional view and an exploded sectional view, respectively, of the steam-sterilizing compartment in accordance with the present invention. Referring to FIGS. 1 to 4, the multi-functional child care storage 100 in accordance with the present invention includes a plurality of functional compartments, i.e., a refrigerating compartment 120, a steam sterilizing compartment 130 and a double-boiling compartment 140 in the present embodiment, in a main body 110 for refrigerating, sterilizing and double-boiling infant products.

The refrigerating compartment 120 includes a refrigerating space 121, and is opened and closed by doors 125 and 126 connected to a main body 110. In the refrigerating space 121, a shelf 122 and a drawer 123 are installed to load infant foods and/or infant products. The refrigerating compartment 120 keeps infant foods and the like loaded on the shelf 122 or in the drawer 123 in a refrigerated state using a thermoelectric element (not shown) or refrigerating machine (not shown).

The steam sterilizing compartment 130 includes a sterilizing space 132 arranged in the main body 110, and is opened and closed from above by a compartment door 131; an evaporation vessel 133 arranged under the base of the sterilizing space 132; a heater 134 arranged under the evaporation vessel 133; an exhaust outlet 135 formed on the compartment door 131; and a blower 136 installed at the compartment door 131.

The sterilizing space 132 is formed at an upper portion of the main body 110, and the compartment door 131 is hinge-jointed to an upper end of the main body 110. As shown in FIGS. 2 to 4, the sterilizing space 132 may be formed inside a sterilization tub 132a, which is detachably installed at the main body 110, so as to be detachable from the main body 110 for cleaning and the like.

A flange 132b is formed at the upper end of the sterilization tub 132a so that the sterilization tub 132a can hang on the main body 110 after being inserted into the main body 110. An opening 132c is formed below the sterilization tub 132a. The evaporation vessel 133 is inserted through the opening 132c from under into the sterilization tub 132a such that the upper portion thereof is exposed to the inside of the sterilization tub 132a. A mount rack 132e is arranged inside the sterilization tub 132a to keep infant products during a sterilization operation.

The evaporation vessel 133 contains water therein for generation of steam, and is inserted into the opening 132c to be located under the sterilizing space 132. A sealing member 133b is disposed between the evaporation vessel 133 and the opening 132c to prevent leakage of the water. A heater groove 133a is formed in the underside of the evaporation vessel 133 to install a heater 134.

In a state where the heater 134 is attached at the heater groove 133a of the evaporation vessel 133, a heater support 134a located under the heater 134 is fixed to the underside of the sterilization tub 132a through fixing members 134b such as screws or bolts. At the underside, the sterilization tub 132a includes fixing portions 132d, to which associated fixing members 134b are coupled. As a result, the heater 134 is interposed between the evaporation vessel 133 and heater support 134a. The heater 134 brought into contact with the evaporation vessel 133 transfers heat to the evaporation vessel 133, and thus the water contained in the evaporation vessel 133 is evaporated into steam.

While various heating elements may be used as the heater 134, a sheath heater is used as the heater 134 in the present embodiment. The sheath heater embeds a heating wire in a vacuum pipe, and has high energy efficiency and good durability against vibration and the like. The sheath heater can also be fabricated in a variety of configurations, facilitating easy installation.

The exhaust outlet 135 is formed at the compartment door 131, through which air in the sterilizing space 132 is exhausted. While the steam sterilizing compartment 130 is not in use, a cover 135a, detachably coupled to the exhaust outlet 135, is used to prevent an inflow of foreign substances.

The blower 136 is installed at the compartment door 131 to generate an air flow for exhausting the air in the sterilizing space 132 to the outside. The compartment door 131 includes a bottom cover 131a and upper cover 131b, which are coupled to each other, to make up a space inside the compartment door 131 and to facilitate installation of the blower 136. In the present embodiment, the blower 136 includes, inside the compartment door 131, a rotatable fan 136a, a motor 136c for generating a driving force, and a belt 136c for linking the shaft of the motor 136c to the fan 136a. The blower 136 may also be configured such that the fan 136a is directly coupled to the shaft of the motor 136c.

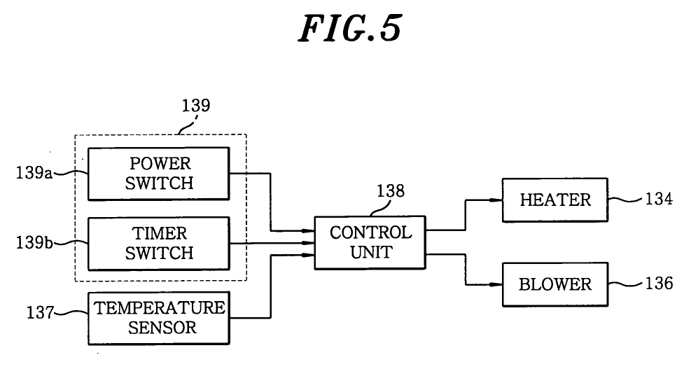

Referring to FIG. 5, there is shown a block diagram illustrating an operation unit, a temperature sensor, a control unit, a heater and a blower in the steam-sterilizing compartment 130 in accordance with the present invention. As shown in FIG. 5, the steam sterilizing compartment 130 further includes a temperature sensor 137 and a control unit 138.

The temperature sensor 137 is installed adjacent to the heater 134, and measures a temperature of the heater 134 and outputs a measurement signal reflecting the measured temperature to the control unit 138.

The control unit 138 receives the measurement signal from the temperature sensor 137, and controls operations of the heater 134 and the blower 136. That is, in response to manipulation of a power switch 139a and a timer switch 139b in an operation unit 139, the control unit 138 controls on/off operations and operating times of the heater 134 and the blower 136. As will be described later, the steam sterilizing compartment sterilizes the infant products loaded therein, and then removes moisture from the infant products. For effective moisture removal after steam sterilization, the blower 136 is controlled to start an operation when the temperature of the heater 134 rises to a preset temperature, for example, ranging from about 105° C. to about 115° C., and preferably, of about 110° C. after the water in the evaporation vessel 133 is completely evaporated. To prevent overheat of the heater 134, the control unit 138 controls the heater 134 to maintain a preset temperature range, for example, from about 95° C. to about 115° C.

Referring again to FIG. 2, for the double-boiling compartment 140, an installation space 143 is provided at an upper portion of the main body 110. The installation space 143 is openable from above by a door 141. A double-boil heater 142 is arranged inside the installation space 143 to provide double-boiling heat. A double-boil tub 144 is detachably inserted into the installation space 143. The double-boil heater 142, adjacent to the outer peripheral surface of the double-boil tub 144, double-boils heated objects such as infant foods and/or infant products, e.g., a milk bottle, contained together with water in the double-boil tub 144.

An insulating material 180 acts to prevent heat transfer between the functional compartments 120, 130 and 140. In addition to the refrigerating compartment 120, steam sterilizing compartment 130 and double-boiling compartment 140, other functional compartments may be provided. For example, a heating compartment may be provided to keep infant foods in a heated state using a heater or a thermoelectric element. A freezing compartment may also be provided to keep infant foods in a frozen state using heat exchange with a coolant. A refrigerating/heating compartment may further be provided to keep infant foods selectively in a refrigerated state or heated state.

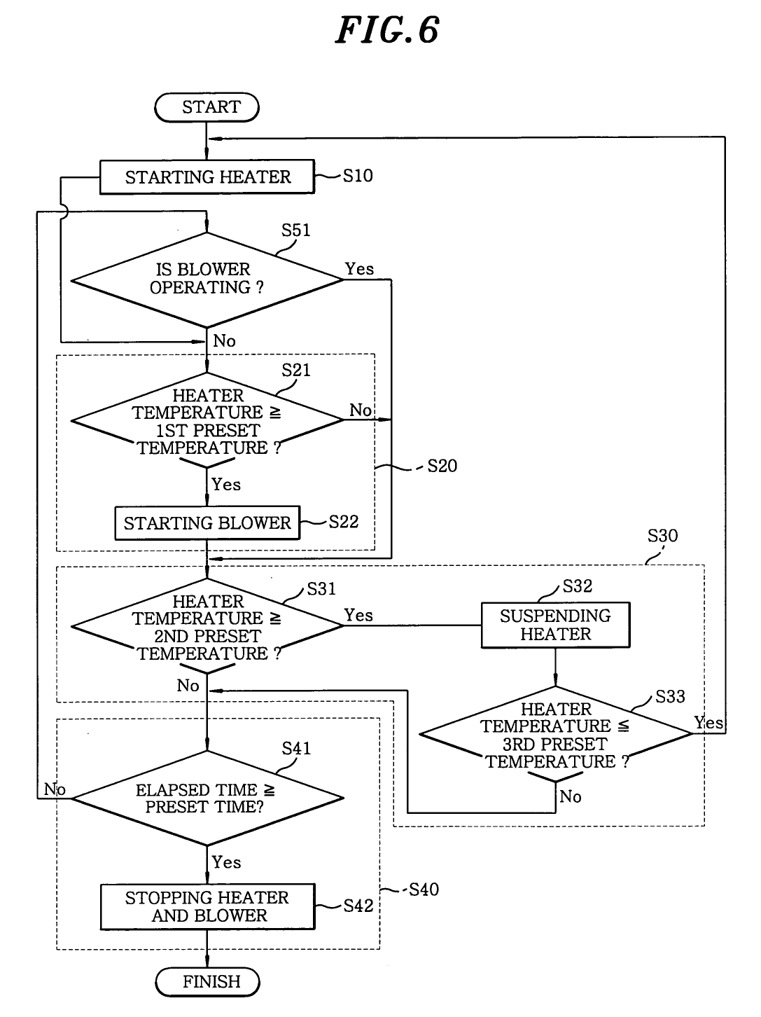

Referring to FIG. 6, a description will be hereinafter given of an operation of the multi-functional child care storage equipped with the steam sterilizing compartment for sterilizing the infant products and then removing moisture therefrom, having the above-described configuration.

Firstly, an infant product to be sterilized is placed on the mount rack 132e of the sterilizing space 132, namely, inside the sterilization tub 132a. The power switch 139a is turned on, and a desired operation time of the heater 134 is set using the timer switch 139b. Hence, the heater 134 starts an operation (S10). With the operation of the heater 134, the water contained in the evaporation vessel 133 is evaporated into hot steam, which sterilizes the infant product in the sterilizing space 132.

Thereafter, the control unit 138 determines whether the blower 136 is in operation (S11). Since, in this case, the blower 136 is not in operation, moisture in the sterilizing space 132 is exhausted to the outside (S20) after complete evaporation of the water in the evaporation vessel 133. That is, after complete evaporation of the water in the evaporation vessel 133, the temperature of the heater 134 comes to rise above 100° C. The control unit 138 determines whether the temperature of the heater 134 measured through the temperature sensor 137 is higher than or equal to a first preset temperature, for example, ranging from about 105 to about 115° C., and preferably, of about 110° C. (S21). If the temperature of the heater 134 is higher than or equal to the first preset temperature, the blower 136 is operated (S22) to exhaust air and moisture in the sterilizing space 132 through the exhaust outlet 135 to the outside, thereby keeping the steam-sterilized infant product in a hygienic dried state.

To prevent overheat of the heater 134 due to the complete evaporation of the water in the evaporation vessel 133, the control unit 138 controls the heater 134 to maintain a preset temperature range (S30). To this end, the control unit 138 determines whether the temperature of the heater 134 is higher than or equal to a second preset temperature of, for example, about 115° C. (S31). If the temperature of the heater 134 is higher than or equal to the second preset temperature, the operation of the heater 134 is suspended (S32). While the operation of the heater 134 is suspended, the control unit 138 determines whether the temperature of the heater 134 is lower than or equal to a third preset temperature of, for example, about 95° C. (S33).

If the temperature of the heater 134 is lower than or equal to the third preset temperature, the operation of the heater 134 is resumed. Then, the control unit 138 determines whether the blower 136 is in operation (S51). If it is determined that the blower 136 is not in operation, step S20 is performed to remove moisture in the sterilizing space 132 through an operation of the blower 136. However, if the blower 136 is in operation, step S20 is skipped to continue the operation of the blower 136, and step S30 is performed to prevent overheat of the heater 134.

While the temperature of the heater 134 is maintained to be lower than the second preset temperature and higher than the third preset temperature through step S30 for the overheat prevention, steam sterilization and moisture exhaustion are performed for a preset time, i.e., an operation time preset by using the timer switch 139b (S40). The control unit 138 determines whether the time elapsed from the initial operation of the heater 134 is longer than or equal to the preset time (S41). If the preset time has expired, the operation of the heater 134 and blower 136 is finished (S42).

As apparent from the above description, the present invention provides a multi-functional child care storage having a steam sterilizing compartment for sterilizing infant products and then removing moisture therefrom. A plurality of functional compartments including the steam sterilizing compartment are provided together to store infant foods and/or infant products in various ways according to their uses and functions so that user's convenience can be enhanced and child care space and time can be saved.

In addition, the steam-sterilizing compartment is used to remove moisture from sterilized infant products with hot air heated by a heater so that the sterilized infant products can be kept in a hygienic state. In the structure of the above-mentioned preferred embodiment, a duct for supplying the hot air is not necessary. As a result, reproduction of germs in the duct while the storage is not in operation can be suppressed, and the structure of the multi-functional child care storage is simplified so that a manufacturing cost thereof can be reduced and the assembly thereof can be made easier.

While the invention has been shown and described with respect to the preferred embodiments, it will be understood by those skilled in the art that various changes and modifications may be made without departing from the scope of the invention as defined in the following claims.

What is claimed is:

1. A multi-functional child care storage comprising a plurality of functional compartments, the plurality of the functional compartments including a steam sterilizing compartment for sterilizing an infant product loaded therein,
wherein the steam sterilizing compartment includes:
a sterilizing space defined for sterilizing the stored infant product;
an evaporation vessel arranged inside the sterilizing space for containing water therein;
a heater installed under the evaporation vessel for heating the evaporation vessel to evaporate the water contained therein into steam;
an exhaust outlet through which air in the sterilizing space is exhausted;
a blower for generating an air flow for exhausting the air in the sterilizing space through the exhaust outlet to the outside; and
a control unit for controlling the blower and the heater to start, suspend or resume operations thereof depending on a temperature of the heater.

2. The multi-functional child care storage of claim 1, wherein the steam sterilizing compartment further includes:
a temperature sensor for measuring the temperature of the heater to generate a measurement signal corresponding to the measured temperature,
wherein the control unit, in response to the measurement signal from the temperature sensor, controls the blower to start an operation thereof when the temperature of the heater rises to a first preset temperature.

3. The multi-functional child care storage of claim 2, wherein the control unit, in response to the measurement signal from the temperature sensor, controls the heater to suspend an operation thereof when the temperature of the heater is higher than or equal to the second preset temperature, and to resume the operation thereof when the temperature of the heater is lower than or equal to the third preset temperature.

4. The multi-functional child care storage of claim 3, wherein the first preset temperature is between about 105° C. and about 115° C.

5. The multi-functional child care storage of claim 4, wherein the second preset temperature is about 115° C. and the third preset temperature is about 95° C.

6. The multi-functional child care storage of claim 3, wherein the sterilizing space is opened and closed from above by a compartment door, the exhaust outlet is formed on the compartment door, and the blower is installed at the compartment door.
